(19) United States
(12) Patent Application Publication
(10) Pub. No.: US 2022/0148708 A1
(43) Pub. Date: May 12, 2022

(54) TECHNOLOGY-FACILITATED SUPPORT SYSTEM FOR MONITORING AND UNDERSTANDING INTERPERSONAL RELATIONSHIPS

(71) Applicants: Shrikanth Sambasivan NARAYANAN, Santa Monica, CA (US); Gayla MARGOLIN, Los Angeles, CA (US); Adela C. AHLE, San Francisco, CA (US); Theodora CHASPARI, College Station, TX (US)

(72) Inventors: Shrikanth Sambasivan NARAYANAN, Santa Monica, CA (US); Gayla MARGOLIN, Los Angeles, CA (US); Adela C. AHLE, San Francisco, CA (US); Theodora CHASPARI, College Station, TX (US)

(73) Assignee: UNIVERSITY OF SOUTHERN CALIFORNIA, LOS ANGELES, CA (US)

(21) Appl. No.: 17/562,647

(22) Filed: Dec. 27, 2021

Related U.S. Application Data
(63) Continuation-in-part of application No. 16/501,103, filed on Mar. 2, 2018.
Application No. 16/501,103 is a continuation-in-part of application No. 16/291,225, filed on Mar. 4, 2019.
(60) Provisional application No. 62/561,938, filed on Sep. 22, 2017.

(51) Int. Cl. G16H 20/70; A61B 5/00; G16H 40/67; A61B 5/16; G06N 3/08

(52) U.S. Cl. CPC G16H 20/70; A61B 5/6801; G16H 40/67; G06N 3/08; A61B 5/6898; A61B 5/165; A61B 5/4803

(57) ABSTRACT

A method for monitoring and understanding interpersonal relationships includes a step of monitoring interpersonal relations of a couple or group of interpersonally connected users with a plurality of smart devices by collecting data streams from the smart devices. Representations of interpersonal relationships are formed for increasing knowledge about relationship functioning and detecting interpersonally-relevant mood states and events. Feedback and/or goals are provided to one or more users to increase awareness about relationship functioning.

### CROSS-REFERENCE TO RELATED APPLICATIONS

This application is a continuation-in-part of U.S. application Ser. No. 16/501,103 filed Mar. 2, 2018, which, in turn, claims the benefit of U.S. provisional application Ser. No. 62/561,938 filed Sep. 22, 2017, the disclosures of which are hereby incorporated in their entirety by reference herein and this application is a continuation-in-part of U.S. application Ser. No. 16/291,225 filed Mar. 4, 2019, the disclosure of which is hereby incorporated in its entirety by reference herein.

### STATEMENT REGARDING FEDERALLY SPONSORED RESEARCH OR DEVELOPMENT

The invention was made with Government support under Contract No. R21 HD072170-A1 awarded by the National Institutes of Health/National Institute of Child Health and Human Development; Contract Nos. BCS-1627272, DGE-0937362, and CCF-1029373 awarded by the National Science Foundation; and Contract No. UL1TR000130 awarded by the National Institutes of Health. The Government has certain rights to the invention.

### TECHNICAL FIELD

In at least one aspect, the present invention is related to automated frameworks for monitoring, quantifying, and modeling interpersonal relationships. In particular, the present invention is related to applications of such frameworks that include the development of novel, individualized measures of relationship functioning and the development of data-driven, automated feedback systems.

### BACKGROUND

The quality of interpersonal relationships is closely tied to both mental well-being and physical health. Frequent conflict in relationships can cause elevated and chronic levels of stress responding, leading to increased risk of cardiac disease, cancer, anxiety, depression, and early death; in contrast, supportive relationships can buffer stress responding and protect health (Burman & Margolin, 1992; Coan, Schaefer, & Davidson, 2006; Grewen, Andersen, Girdler, & Light, 2003; Holt-Lunstad, Smith, & Layton, 2010; Leach, Butterworth, Olesen, & Mackinnon, 2013; Robles & Kiecolt Glaser, 2003). Epidemiological research has shown that the health risks of social isolation are comparable to other well-known risk factors, such as smoking and lack of exercise (House, Landis, & Umberson, 1988). Other research shows that family relationships, including the way parents interact with their children, have a large impact on child functioning across the lifespan, contributing to the development of psychological problems, as well as poor health outcomes in adulthood (Springer, Sheridan, Kuo, & Carnes, 2007). More broadly, research suggests that other types of interpersonal stressors, such as conflicts with coworkers, are highly stressful, impact our physical and mental health, and contribute to missed workdays and decreased well-being (Sonnentag, Unger, & Nagel, 2013). The toll of negative relationships on physical and mental health, taken in combination with lost productivity at work, results in billions of dollars of lost revenue annually (Lawler, 2010; Sacker, 2013).

To date, attempts to detect psychological, emotional, or interpersonal states via machine learning and related technologies have largely been done in controlled laboratory settings, for example identifying emotional states during lab-based discussion tasks (e.g., Kim, Valente, & Vinciarelli, 2012; Hung, & Englebienne, 2013). Other research has attempted to automatically detect events of interest in uncontrolled settings as people live out their daily lives; however, these attempts have focused on detecting discrete and more easily identifiable states, e.g., whether people are exercising versus not exercising, or have pertained to individuals rather than systems of people (Lee et al., 2013). Although a small number of researchers have attempted to use machine learning and wearable sensing technology to detect and even predict psychologically-relevant states in daily life, these projects have focused almost exclusively on detecting individual mood states and behaviors, rather than modeling dynamic, interpersonal processes (Bardus, Hamadeh, Hayek, & Al Kherfan, 2018; Comello & Porter, 2018; Farooq, McCrory, & Sazonov, 2017; Forman et al., 2018; Knight & Bidargaddi, 2018; Knight et al., 2018; Pulantara, Parmanto, & Germain, 2018; Rabbi et al., 2018; Sano et al., 2018; Skinner, Stone, Doughty, & Munafo, 2018; Taylor, Jacques, Ehimwenma, Sano, & Picard, 2017; Vinci, Haslam, Lam, Kumar, & Wetter, 2018). No research to our knowledge has used machine learning to model interpersonal relationships in real life via wearable technologies. Detecting complex emotional and interpersonal states, e.g., feeling close to someone or having conflict, in real life settings is difficult because there is substantially more variability in the data, where various confounding factors, e.g., background speech, could influence signals and decrease the accuracy of the identification systems.

Accordingly, there is a need for improved methods and systems for monitoring and improving interpersonal relationships.

### SUMMARY

The present invention solves one or more problems of the prior art by providing in at least one embodiment, a method and system for improving the quality of relationship functioning. The system is advantageously compatible with various technologies—including but not limited to smartphones, wearable devices that measure a user's activities and physiological state (e.g., Fitbits), smartwatches, other wearables, and smart home devices—that makes use of multimodal data to provide detailed feedback and monitoring and to improve relationship functioning, with potential downstream effects on individual mental and physical health. Using pattern recognition, machine learning algorithms, and other technologies, this system detects relationship-relevant events and states (e.g., feeling stressed, criticizing your partner, having conflict, having physical contact, having positive interactions, providing support) and provides tracking, monitoring, and status reports. This system applies to a variety of relationship types, such as couples, friends, families, workplace relationships, and can be employed by individuals or implemented on a broad scale by institutions and large interpersonal networks, for example in hospital or military settings.

In another embodiment, a method for monitoring and understanding interpersonal relationships is provided. The method includes a step of monitoring interpersonal relations of a couple or group of interpersonally connected users with a plurality of smart devices by collecting data streams from the smart devices. The interpersonal relations are classified and/or quantified into classification or quantifications. Feedback and/or goals are provided to the couple or group of interpersonally connected users to increase awareness about relationship functioning.

### BRIEF DESCRIPTION OF THE DRAWINGS

FIG. 1A is a schematic of a framework implementing embodiments of the invention.

FIG. 1B is a schematic of a smart device used in the system of FIG. 1A.

FIG. 2A is a screen view providing the user a progress bar giving an indication of interpersonal relation improvement.

FIG. 2B is a screen view of a login screen.

FIG. 2C is a screen view that provides feedback scores and other indicators for relationship characteristics.

FIG. 2D is a screen view that provides feedback scores and other indicators for relationship characteristics.

FIG. 2E is a screen view that provides feedback scores and other indicators for relationship characteristics.

FIG. 2F is a screen view that provides feedback scores and other indicators for relationship characteristics.

FIG. 2G is a screen view that provides feedback scores and other indicators for relationship characteristics.

FIG. 2H is a screen view that provides feedback scores and other indicators for relationship characteristics.

FIG. 2I is a screen view that provides metrics with respect to progress in improving interpersonal relations.

FIG. 2J is a screen view that provides metrics with respect to progress in improving interpersonal relations.

FIG. 3 a diagram of exemplary representations of interpersonal relationships such as attachment, emotion regulation, and enmity.

FIG. 4A is a schematic representation of a classification system. Multimodal classification (task 3) between conflict and non-conflict samples (S1 . . . SN) used combinations of features from each modality: self-reported mood and quality of interactions (MQI), electrodermal activity (EDA), electrocardiogram (ECG) activity, EDA synchrony measures, language, and acoustic information.

FIG. 4B is an example of a decision tree.

FIG. 5. Schematic illustrating a method in which personal interactions are evaluated and classified by considering clusters of users with similar traits.

FIG. 6. Receiver operating characteristic (ROC) curves for self-reported mood and quality of interaction (such as stressed, happy, sad, nervous, angry, and close) and multimodal feature groups. Multimodal-F-a=female EDA, psychological, personal, interaction, context; Multimodal-F-b=female self-reported MQI, EDA, psychological, personal, acoustic, interaction, context; Multimodal-M-a=male ECG activity, psychological, Acoustic, interaction, context; Multimodal-M-b=male self-reported MQI, EDA synchrony, paralinguistic, personal, acoustic, interaction, context.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

Throughout this application, where publications are referenced, the disclosures of these publications in their entireties are hereby incorporated by reference into this application to more fully describe the state of the art to which this invention pertains.

In an embodiment, a method of monitoring and understanding interpersonal relationships is provided. The method includes a step of monitoring interpersonal relations for a couple or a group of interpersonally connected users with a plurality of smart devices by collecting data streams from the smart devices. Typically, the data streams are obtained directly from the smart device or from wearable sensors worn by the couple or group of interpersonally connected users and in communication (e.g., wireless or wired) with the smart devices. Typically, the smart devices are mobile smart devices. Examples of smart devices include, but are not limited to, smart phones (e.g., iPhones), tablet computers (e.g., iPads), and the like. In the context of the present embodiment, monitoring means receiving data generated from the smart devices and/or sensor in communication with the smart devices. Classification and/or quantification of the interpersonal relations are determined from the monitoring and optionally used to form representations of interpersonal relationships. In this context, representation means an assigned descriptor (e.g., a general category) of an interpersonal relationship that can include one or more characteristics that can describe the interpersonal relationship. In a refinement, descriptor and/or the related classifications can be binary or continuous. For example, the presence or absence of conflict may be characterized as present or not present (i.e., binary). In contrast, a characteristic such as mood may be quantified by a continuous parameter.

The step of classifying and/or quantifying of the interpersonal relations is achieved from predetermined classification groups or correlations that are obtained from test data with known classifications or quantifications (e.g., correlations). For example, a continuous feature such as positive mood might be quantified by a number where the larger the number the more positive the mood. This step can be achieved by machine learning techniques that are described below in more detail. In a variation, signal-derived features are extracted from the data streams. The signal derived features provide inputs to a trained neural network that determined interpersonal classifications that allow selection of a predetermined feedback to be sent.

In one variation, the method can be implemented within a smart device in the possession of a user. An app on the smart device can provide real-time monitoring of interactions by receiving the data streams from the user in possession of the device and from other users. Feedback as required can be provided by the app. In another variation, the data streams are received by and/or the interventions signals generated by a monitor smart device.

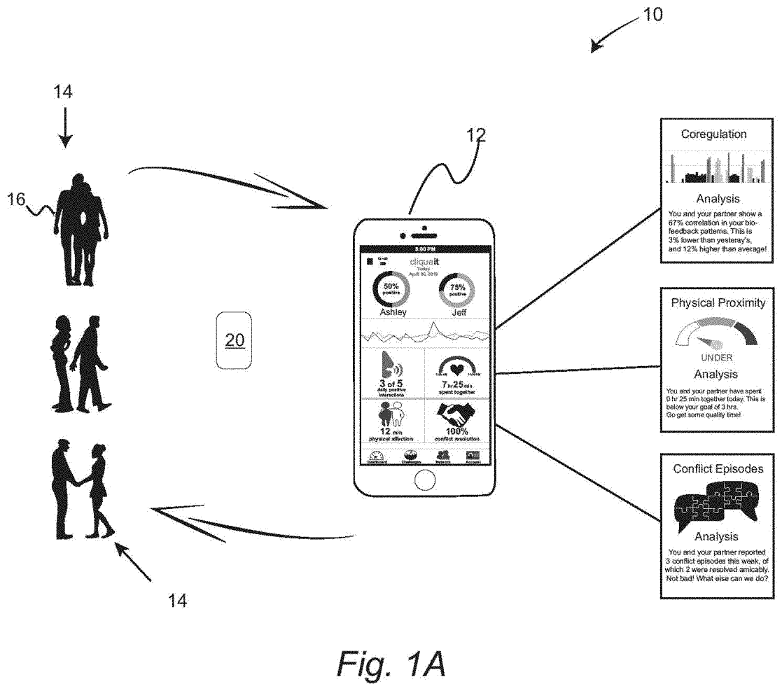

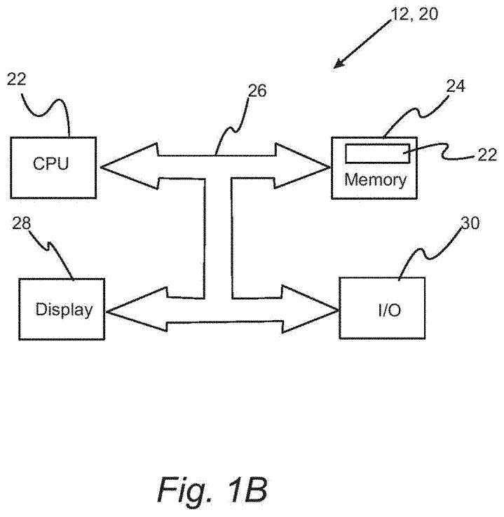

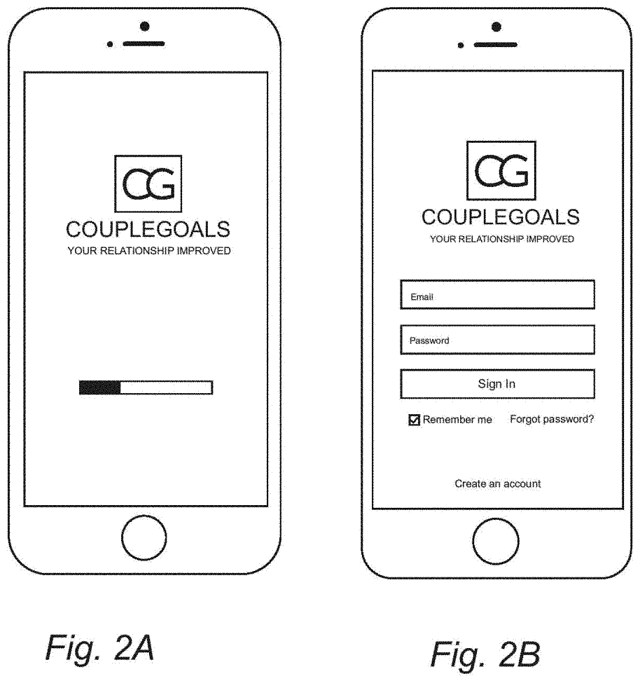

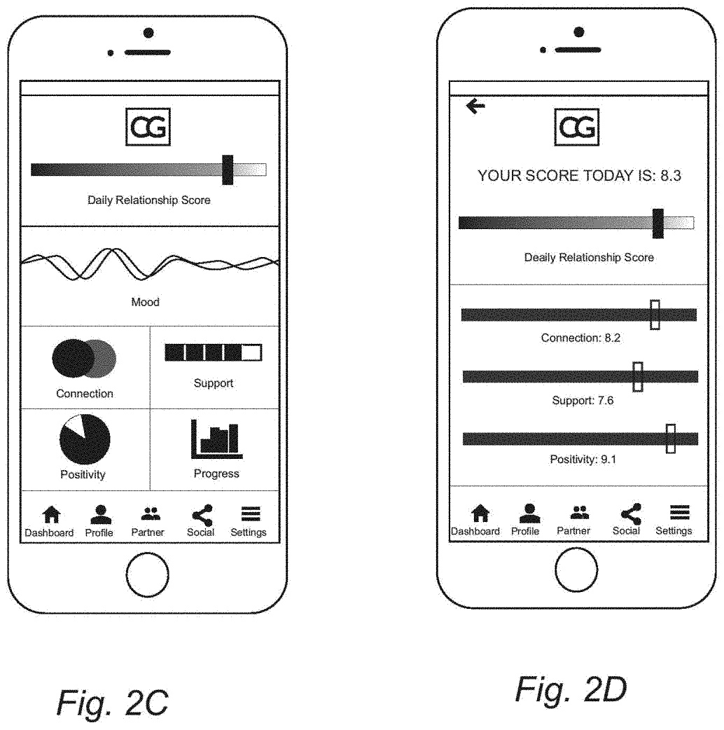

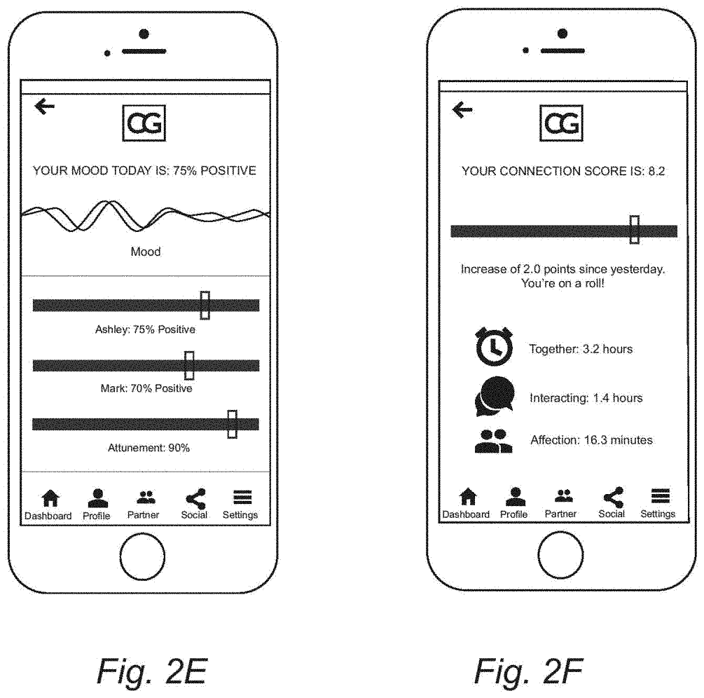

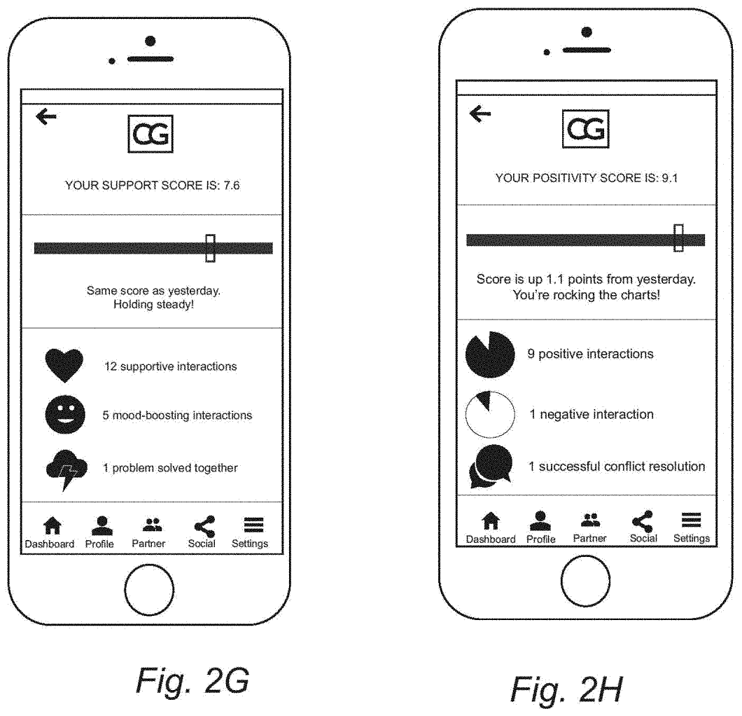

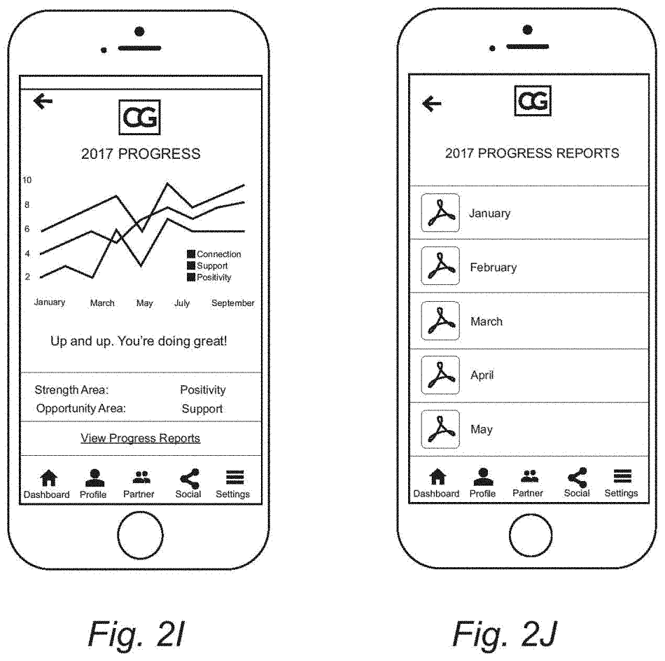

In a refinement as set forth below in more detail, classification of the interpersonal relations can be achieved by applying various machine learning techniques (e.g., neural networks, decision tress, state vector machines, and combinations thereof). The representations can increase knowledge about relationship functioning and determine interpersonally-relevant mood states and events. Examples of such representations include general categories such as attachment, emotion regulation, and enmity, which could include the frequency of positive interactions between people in relationships, feelings of closeness, and the amount of quality time spent together (e.g., minutes spent together and interacting), mood of each person, covariation or coregulation mood (how synchronous people are in mood states and how they mitigate negative mood in each other), stress contagion (how negative mood in one person transfers to another). FIGS. 1A, 1B which are described below in more detail provide illustrations of systems that implement the present method of monitoring and understanding interpersonal relationships. FIGS. 2A-2J provide smart phone screens that users interact with in practicing the method of monitoring and understanding interpersonal relationships.

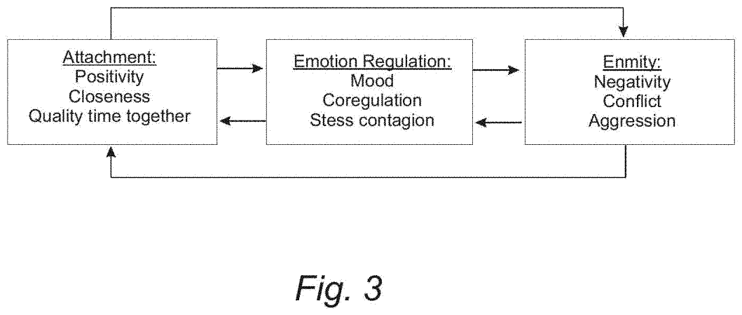

With reference to FIG. 3, a diagram of exemplary representations of interpersonal relationships such as attachment, emotion regulation, and enmity is provided. In a refinement, feedback and/or goals are provided to one or more users to increase awareness about relationship functioning. Goals could include amplifying attachment bonds, increasing positivity, increasing closeness, increasing quality time spent together (defined as time together and interacting), boosting levels of positive mood and decreasing levels of negative mood, improving emotion regulation skills, and decreasing the amount of aggression and conflict. The feedback allows users to meet these goals by encouraging them to alter behavior as needed.

In a variation, the representations of interpersonal relationships can be signal-derived and/or machine-learning based representations. In a refinement, the data streams include one or more components selected from the group consisting of physiological signals (e.g. blood volume pulse, electrodermal activity, electrocardiogram, respiration, acceleration, body temperature as measured by wearable sensors or devices such as Fitbits, Apple watches, EMPATICA™ E4, Polar T31 ECG belt, etc.); audio measures; speech content; video; GPS; light exposure; content consumed and exchanged through mobile; internet; network communications; sleep characteristics; interaction measures between individuals and across channels such as time spent interacting face-to-face or remotely; frequency of conflicts; physiological, acoustic, and linguistic co-regulation; and self-reported data about relationship quality, negative and positive interactions, and mood. In a refinement, pronoun use, negative emotion words, swearing, certainty words in speech can be collected from the smart devices and evaluated. In a refinement, the evaluation can be implemented by machine learning techniques such as neural networks, decision trees, and combinations thereof. In another refinement, sleep length or quantity can be quantified. The content of text messages and emails, time spent on the internet, number or length of tests and phone calls in the network communications can also be collected from the smart devices, measured, and evaluated by machine learning techniques (e.g., neural networks, decision trees, and combinations thereof). In the context of the present embodiments, variations, and refinements, evaluation includes the process of classification as described below in more detail.

The data collected in the variations and refinements set forth above can be stored separately in a peripheral device or integrated into a single platform. Examples of suitable peripheral storage devices include, but are not limited to, a wearable sensor, cell phone, or audio storage device. In another refinement, the single platform can be a mobile device or IoT platform.

In a variation, the method further includes a step of computing signal-derived features from the data streams. Such signal-derived features are suitable as inputs to machine learning techniques such as neural networks. Acoustic features include motor timing parameters of speech production (e.g., speaking rate and pause time), prosody and intonation (e.g., loudness and pitch), and frequency modulation (e.g., spectral coefficients). Linguistic features include word count, frequency of parts of speech (e.g., nouns, personal pronouns, adjectives, verbs), frequency of words related to affect, stress, mood, family, aggression, work. Physiological measures include skin conductance level, mean skin conductance response frequency and amplitude, rise and recovery time of skin conductance responses, average, standard deviation, minimum, and maximum of the inter-beat interval (IBI), average beats per minute, heart rate variability, R-R interval, as well as the very-low (<0.04 Hz), low (0.04-0.15 Hz), and high (0.15-0.4 Hz) frequency component of the IBI, breathing rate, breathing rate variability, mean acceleration, acceleration entropy, and/or Fourier coefficients of acceleration. Similarity measures between the two partners can be computed with respect to the above features in order to integrate momentary co-regulation as a feature to the machine learning system. In addition to that, raw signals could be used as features (e.g., inputs) for the machine learnings algorithms, which will learn feature transformations for the outcome of interest. Custom made toolboxes available online and developed in the lab can be used to derive such metrics. The signal-derived representation can be computed by knowledge-based design and/or data-driven analyses, which can include clustering. For example, all of these data (raw signal and extracted measures) can be used as features for algorithm development where the ground truth is established via either or a combination of self-report data from concurrent phone surveys or through observational codes obtained from audio and/or video data. Ground truth constructs (e.g., conflicts) can then be used as labels in algorithms to detect the identified target states for monitoring. In the context of the present invention, the term “algorithm” includes any computer-implemented method that is used to perform the methods of the invention. In particular, machine learning algorithms include a variety of models, such as neural networks, support vector machines, binary decision trees, and the like. Leave-one-out cross validation would be conducted to assess accuracy. Standard evaluation metrics can be applied (e.g., kappa, F1-score, mean absolute errors). In addition to supervised learning methods, unsupervised methods could be used to detect clusters in the data that were not hypothesized beforehand (e.g., supervised and unsupervised neural networks). Theory-based models could include using questionnaire or other data to build subpopulation specific models for increased accuracy (e.g., subpopulation models built on aggression levels).

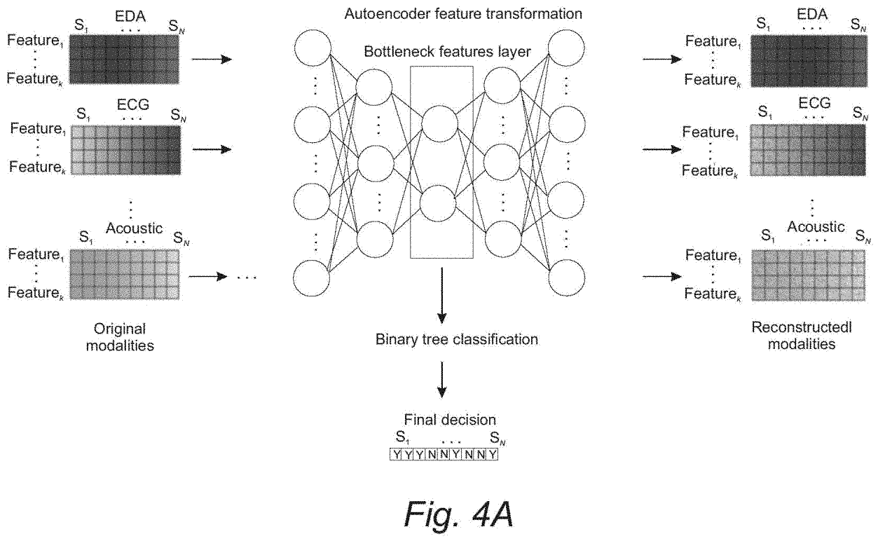

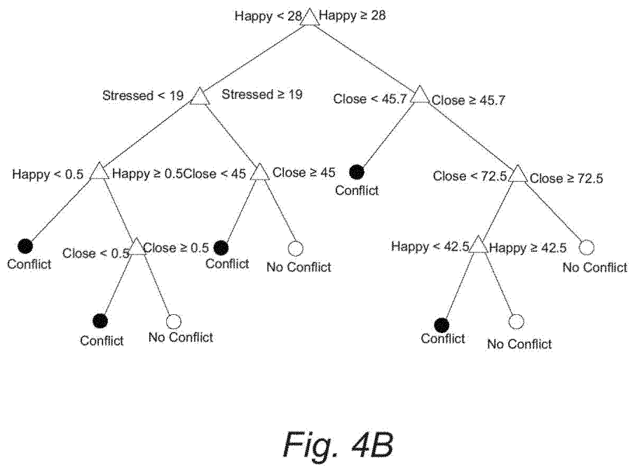

In a refinement, the signal-derived features (e.g., signal derived representations) are used as inputs for machine learning, data mining, and statistical algorithms that can be used to determine what factors, or combinations of factors, predict the classification of a variety or relationship dimensions, such as conflict, relationship quality, or positive interactions. This means machine learning will be used to detect all the state constructs of interest via a variety of algorithms (e.g., neural networks, support vector machine, and the like). Furthermore, one can obtain data on relationship functioning over time (e.g., from questionnaire data or from the phone-based metrics) to determine which families or couples (or other systems of people) are experiencing increases or decreases in relationship functioning (e.g., decreases in conflict). Machine learning methods can then be used to retroactively determine what features or combinations thereof predict changes in relationship functioning over time. FIG. 4A provides an example of a procedure for training an algorithm which know classifications (e.g., form self-reporting) to be used in classification. A combination of self-report data, coding of interviews, observations, videos, and/or audio recordings can be compared to the signal-derived features to determine the accuracy of the systems. In FIG. 4A, an autoencoder neural network is trained with features (e.g., feature₁ . . . featureₖ) as input modalities characterizing each of a variety of signals (e.g., EDA signals, ECG signals, Acoustic signals, and the like). The objective of the autoencoder is to learn a reduced representation of the original feature set. The reduced feature set is obtained from the bottleneck layer, which is learned so that the right side of the autoencoder network can generate a representation as close as possible to the original signal from the reduced encoding. From the bottleneck layer, inputs for drafting a binary decision tree are extracted. The output at the right of FIG. 4A provide reconstructed modalities which should provide a good approximation of the inputs if the neural network has been properly trained. Once trained, the neural network and the decision tree can be used to perform the classification. FIG. 4B is an example of a decision tree. In a variation, statistics, e.g., regression analyses, latent class analysis can be used to predict changes in relationship functioning.

In another variation, individual models are used to increase classification accuracy since patterns of interaction may be specific to individuals, couples, or groups of individuals. This could involve building sub-population specific machine learning models. Models would leverage common information across all people and then fine-tune decisions based on the sub-population (e.g., level of aggression in the relationship, level of depression, sociodemographic factors) of interest. Decisions will be made for clusters of people with common characteristics, improving accuracy and reducing the amount of data needed for training. Models could use multi-task learning to leverage useful information from related sub-populations. By operationalizing multi-task learning as a feature-learning approach, it can be assumed that people share some general feature representations, while specific representations can later be learned for every subpopulation. Multi-task learning could be implemented using deterministic and probability methods (i.e., train the first layers of the feedforward neural networks based on the entire dataset to represent common feature embeddings and then refine the last layers for each subpopulation separately).

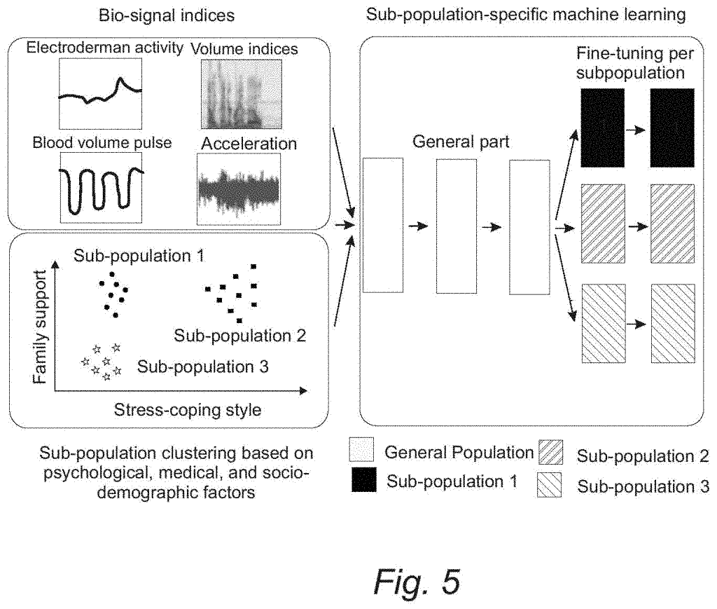

In a refinement, active and semi-supervised learning methods are applied to increase predictive power as people continue to use a system implementing the method of monitoring and understanding interpersonal relationships. For example, reinforcement learning models could be used to determine whether to administer a phone survey at a given time. The goal of the algorithm would be to solve a sequential decision problem, where at each stage there are two possible actions (to administer or not administer the phone survey). The algorithm would attempt to achieve optimal balance between maximizing a cumulative reward function (i.e., correct identifying a target state such as conflict) and exploring unseen regions of the input (i.e., administering a phone survey for exploring bio-signal patterns that have not yet been observed). The state-space of the algorithm would be represented by the bio-signals, estimates from the sub-population specific machine learning models, and time elapsed since the last phone survey (to prevent the continuous administration of phone surveys). Receiving estimates from subpopulation specific machine learning algorithms will prevent the reinforcement learning algorithms from exploring too many irrelevant bio-signal patterns in the first steps. The reward function would be the person's response to the phone survey over previous time points with similar bio-signal patterns to the current one. Similarity could be computed via a distance measure (e.g., Euclidean norm) between the current and previous bio-signal indices in past phone surveys. The current action would reflect the trade-off between maximizing the cumulative reward (i.e., observing the state) and adequately exploring unseen feedbacks (i.e., a bio-signal pattern for which a phone survey has not been received before). If the algorithm decides to administer a survey, the reward function will be populated with an additional pair of values and the cumulative reward will be updated based on the self-reported data. The goal of this algorithm (e.g., 1-armed bandit) would be to gradually learn each person's state-related bio-signals over time and to administer phone surveys only when the state is detected. FIG. 5 provides an example of a clustering algorithm in which users are grouped by similar characteristics (e.g., family support, stress coping style, and the like). A neutral network is used to provide inter-group classifications (e.g., criteria of classifications is group specific).

As set forth above, characterization of the interpersonal relations can be performed by a neural network that receives by the data collecting from the smart phones as inputs. The neural network can be trained from previously obtained data (e.g., the signal derived features set forth above) that has an assigned know classification which can be obtained by users self-reporting (e.g., stating they are in conflict). As set forth below in the examples, the neural network can be used to provide training inputs to a decision tree. The computer tree can be alternatively be used (e.g., instead of using a neural network) to perform the classify step set forth above.

In another variation, the relationship functioning includes indices selected from the group consisting of a ratio of positive to negative interactions, number of conflict episodes, an amount of time two users spent together, an amount of quality time two users spent together (i.e., time spent interacting and speaking with each other), amount of physical contact, exercise, time spent outside, sleep quality and length, and coregulation or linkage across these measures. These metrics are determined by self-report phone-based surveys and coded observational data from audio and/or video recordings. In a refinement, the method further includes a step of suggesting goals for these indices and allowing users to customize their goals.

In some variations, feedback can be provided as ongoing tallies and/or graphs viewable on the smart device. This could include information about changes in relationship functioning, number of conflicts, number of positive interactions, etc. FIGS. 2I and 2J provide an example of smart phone screen views that provide such tallies to users with a smart device. In a refinement, daily, weekly, and yearly reports of relationship functioning are created. In another refinement, the users can view, track, and monitor each of these data streams and their progress on their goals via customizable dashboards.

In a variation, the method further includes a step of analyzing each data stream to provide a user with covariation of user's mood, relationship functioning, and various relationship-relevant events. Such metrics are obtained via time series analysis and dynamical systems model (DSM) coefficients quantifying the association and influence of each person on the other person (i.e., one person's mood impacting the other person's mood). Emotional escalation patterns within and individual and between individuals can be quantified though a DSM, such as a coupled linear oscillator incorporating each individual's emotional arousal (as depicted from physiological, acoustic, and linguistic indices), and the effect of the interacting partner on these measures. The DSM parameters reflect the amount of emotional self-regulation within a person, as well as within-couple co-regulation. The user can also create personalized networks and specify relationship types for each person in their network. This means that there could be multiple people in an interpersonal system that are linked in the system as a network (e.g., someone linked as mother, someone linked as child, someone linked as coworker, someone linked as friend). It would thus be possible to compute these metrics for dyads or systems of people within the network, rather than just between one dyad using the system. The user can also set person-specific privacy settings and customize personal data that can be accessed by others in their networks.

With reference to FIGS. 1A, 1, and 2, a system that implements the previously described method of monitoring and understanding interpersonal relationships is provided. System 10 includes a plurality of mobile smart devices 12 operated by a plurality of users 14. In a variation, the system can further include a plurality of sensors 16 (e.g., heart rate sensors, blood pressure sensors, etc. as set forth above) worn by the plurality of users. In a refinement as set forth above, the data streams are received by and/or the feedback signals generated by a monitor smart device 20. Device 20 may also perform the classification. The mobile smart devices 12 and optionally monitor smart device 20 used this system that implements the abovementioned method can include a microprocessor and a non-volatile memory on which instructions for implementing the method are stored. For example, smart devices 12, 20 can include computer processor 22 that executes one, several, or all of the steps of the method. It should be appreciated that virtually any type of computer processor may be used, including microprocessors, multicore processors, and the like. The steps of the method typically are stored in computer memory 24 and accessed by computer processor 22 via connection system 26. In a variation, connection system 26 includes a data bus. In a refinement, computer memory 24 includes a computer-readable medium which can be any non-transitory (e. g., tangible) medium that participates in providing data that may be read by a computer. Specific examples for computer memory 24 include, but are not limited to, random access memory (RAM), read only memory (ROM), hard drives, optical drives, removable media (e.g. compact disks (CDs), DVD, flash drives, memory cards, etc.), and the like, and combinations thereof. In another refinement, computer processor 22 receives instructions from computer memory 24 and executes these instructions, thereby performing one or more processes, including one or more of the processes described herein. Computer-executable instructions may be compiled or interpreted from computer programs created using a variety of programming languages and/or technologies including, without limitation, and either alone or in combination, Java, C, C++, C#, Fortran, Pascal, Visual Basic, Java Script, Perl, PL/SQL, etc. Display 28 is also in communication with computer processor 22 via connection system 16. Smart device 12, 20 also optionally includes various in/out ports 30 through which data from a pointing device may be accessed by computer processor 22. Examples for monitor device 20 include, but are not limited to, desktop computers, smart phones, tablets, or tablet computers.

FIGS. 2A-2J provide smart phone screens that users interact with in practicing the methods of the invention. FIG. 2A is a screen view providing the user a progress bar giving an indication of interpersonal relation improvement. FIG. 2B is a screen view of a login screen. FIGS. 2C, 2D, 2E, 2F, 2G, and 2H are screen view providing feedback scores and other indicators for relationship characteristics such as relationship score, connection, support and positivity. FIGS. 2I and 2J are screen views providing metrics with respect to progress in improving interpersonal relations.

The following examples illustrate the various embodiments of the present invention. Those skilled in the art will recognize many variations that are within the spirit of the present invention and scope of the claims.

Prototype Model

Using mobile computing technology, our field study collected self-reports of mood and the quality of interactions (MQI) between partners, EDA, ECG activity, synchrony scores, language use, acoustic quality, and other relevant data (such as whether partners were together or communicating remotely) to detect conflict in young-adult dating couples in their daily lives. We conducted classification experiments with binary decision trees to retro actively detect the number of hours of couple conflict.

To assess our approach's usefulness, our study addressed four interrelated research questions that generated four tasks:

Question 1: Are theoretically driven features related to conflict episodes in daily life? Task 1: We conducted individual experiments for theoretically driven features, including self-reported MQI, EDA, ECG activity, synchrony scores, personal pronoun use, negative emotion words, certainty words, F0, and vocal intensity.

Question 2: Are unimodal feature groups related to conflict episodes in daily life? Task 2: We combined the features into unimodal groups to determine the classification accuracy of different categories of variables.

Question 3: Are multimodal feature combinations related to conflict episodes in daily life? Task 3: We combined the feature groups into multimodal indices to examine the performance of multiple sensor modalities.

Question 4: How do multimodal feature combinations compare with the couples' self-report data? Task 4: We statistically compared the classification accuracy of our multimodal indices to the couples' self-reported MQI to ascertain the potential of these methods to identify naturally occurring conflict episodes beyond what participants themselves reported, hour by hour.

Our objective here is to present preliminary data and demonstrate our classification system's potential utility for detecting complex psychological states in uncontrolled settings. Although this study collected data on dating couples, these methods could be used to study other types of relationships, such as friendships or relationships between parents and children.

Research Methodology

The participants in our study consisted of young-adult dating couples from the Couple Mobile Sensing Project, with a median age of 22.45 years and a standard deviation (SD) of 1.60 years. The couples were recruited from the greater Los Angeles area and had been in a relationship for an average of 25.2 months (SD=20.7). Participants were ethnically and racially diverse, with 28.9 percent identifying as Hispanic, 31.6 percent Caucasian, 13.2 percent African-American, 5.3 percent Asian, and 21.1 percent multiracial.

Out of 34 couples who provided data, 19 reported experiencing at least one conflict episode and thus were included in the classification experiments. All study procedures were approved by the USC Institutional Review Board.

Measures

All dating partners were outfitted with two ambulatory physiological monitors that collected EDA and ECG data for one day during waking hours. They also received a smartphone that alerted them to complete hourly self-reports on their general mood states and the quality of their interactions. The self-report options, which were designed to assess general emotional states relevant to couple interactions, included feeling stressed, happy, sad, nervous, angry, and close to one's partner. Responses ranged from 0 (not at all) to 100 (extremely).

Additionally, each phone continuously collected GPS coordinates, as well as 3-minute audio recordings every 12 minutes from 10:00 a.m. until the couples went to bed.

Physiological indices. We collected physiological measures continuously for one day, starting at 10:00 a.m. and ending at bedtime. EDA, activity count, and body temperature were recorded with a Q-sensor, which was attached to the inside of the wrist using a band. ECG signals were collected with an Actiwave, which was worn on the chest under the clothing. ECG measures included the interbeat interval (IBI) and heart rate variability (HRV), and EDA features consisted of the skin conductance level (SCL) and the frequency of skin conductance responses (SCRs). Estimates of synchrony, or covariation in EDA signals between romantic partners, were obtained using joint-sparse representation techniques with appropriately designed EDA-specific dictionaries. (T. Chaspari et al., 2015; the entire disclosure of which is hereby incorporated by reference).

We used computer algorithms to detect artifacts, which were then visually inspected and revised. All scores were averaged across each hour to obtain one estimate of each measure per hour-long period.

Language and acoustic feature extraction. A microphone embedded in each partner's smartphone recorded audio during the study period. The audio clips were 3 minutes long and collected once every 12 minutes, resulting in 6 minutes of audio per 12 minutes per pair (male and female within a couple). This resulted in a reasonable tradeoff between the size of the audio data available for storage and processing and the amount of acquired information. For privacy considerations, participants were instructed to mute their microphones when in the presence of anyone not in the study.

We transcribed and processed audio recording using Linguistic Inquiry and Word Count (LIWC) software. (Pennebaker, 2007; the entire disclosure of which is hereby incorporated by refernce). For our theoretically driven features (task 1), we used preset dictionaries representing personal pronouns (such as “I” and “we”), certainty words (such as “always” and “must”), and negative emotion words (such as “tension” and “mad”). To test unimodal combinations of features, we used four preset LIWC categories, including linguistic factors (25 features including personal pronouns, word count, and verbs), psychological constructs (32 features such as words relating to emotions and thoughts), personal concern categories (seven features such as work, home, and money), and para linguistic variables (three features such as assents and fillers).

Voice-activity detection (VAD) was used to automatically chunk continuous audio streams into segments of speech or nonspeech. We used speaker clustering and gender identification to automatically assign a gender to each speech segment. We then extracted vocally encoded indices of arousal (F0 and intensity). To map the low-level acoustic descriptors onto a vector of fixed dimensionality—independent of the audio clip duration—we further computed the mean, SD, maximum value, and first-order coefficient of the linear regression curve over each speech segment, resulting in eight features. All acoustic and language features were calculated separately by partner and averaged per hour.

Context and interaction indices. In addition to our vocal, language, self-reported, and physiological variables, we assessed numerous other factors that are potentially relevant for identifying conflict episodes. The contextual variables included whether participants consumed caffeine, alcohol, tobacco, or other drugs; whether they were driving; whether they exercised; body temperature; and physical activity level. The interactional variables involved the GPS-based distance between partners and information related to whether the dating partners were together, interacting face to face, or communicating via phone call or text messaging and if they were with other people.

The data for the contextual and interactional feature groups were collected via various mechanisms, including physiological sensors, smartphones, self-reports based on the hourly surveys, and interview data.

Conflict. We identified the hours in which conflicts occurred using the self-report phone surveys. For each hour, participants reported whether they “expressed annoyance or irritation” toward their dating partner using a dichotomous yes/no response option. Because determining what constitutes a conflict is subjective, we elected to use a discrete behavioral indicator (that is, whether the person said something out of irritation) as our ground-truth criterion for determining if conflict behavior occurred within a given hour. This resulted in 53 hours of conflict behavior and 182 hours of no conflict behavior for females and 39 hours of conflict behavior and 206 hours of no conflict behavior for males.

Conflict Classification System

The goal of the classification task was to retroactively distinguish between instances of conflict behavior and no conflict behavior, as reported by the participants. The analysis windows constituted nonoverlapping hourly instances starting at 10:00 a.m. and ending at bedtime.

To classify conflict, we used a binary decision tree because of its efficiency and self-explanatory structure. We employed a leave-one-couple-out cross-validation setup for all classify-cation experiments. For tasks 2 and 3, feature transformation was performed through a deep autoassociative neural network, also called an autoencoder, with three layers in a fully unsupervised way. The autoencoder's bottle neck features at the middle layer consisted of the input of a binary tree for the final decision (Y=conflict and N=no conflict). Unimodal classification followed a similar scheme, under which the autoencoder transformed only the within-modality features. FIG. 4 presents a schematic representation of the classification system as it applies to our dataset.

Further details regarding the system, a list of the entire feature set, complete results from all our experiments, and confusion matrices (that is, tables showing the performance of the classification model) are available online at homedata.github.io/statistical-methodology.html.

Results

Our study results showed that several of our theoretically driven features (such as self-reported levels of anger, HRV, negative emotion words used, and mean audio intensity) were associated with conflict at levels significantly higher than chance, with an unweighted accuracy (UA) reaching up to 69.2 percent for anger and 62.3 percent for expressed negative emotion (task 1). This initial set of results is in line with laboratory research linking physiology and language use to couples' relationship functioning. (Amato, 2000; Levenson and Gottman, 1985; Timmons, Margolin, and Saxbe, 2015; Simmons, Gordon, and Chambless, 2005; Baucom, 2012). When testing unimodal feature groups (task 2), the levels of accuracy reached up to 66.1 and 72.1 percent for the female and male partners, respectively. Combinations of modalities based on EDA, ECG activity, synchrony scores, language used, acoustic data, self-reports, and context and interaction resulted in UAs up to 79.6 percent (sensitivity=73.5 percent and specificity=85.7 percent) for females and 86.8 percent (sensitivity=82.1 percent and specificity=91.5 percent) for males. Using all features except self-reports, the UA reached up to 79.3 percent. These findings generally indicate that it is possible to detect a complex, psychological state with reasonable accuracy using multi-modal data obtained in uncontrolled, real-life settings.

Because we aim to eventually detect conflict using passive technologies only—that is, without requiring couples to complete self-report surveys—we compared the UAs based only on self-reported MQI to combinations incorporating passive technologies (task 4). These results showed several setups where multimodal feature groups with and without self-reported MQI data significantly exceeded the UA achieved from MQI alone. This indicates that the passive technologies added predictability to our modeling schemes.

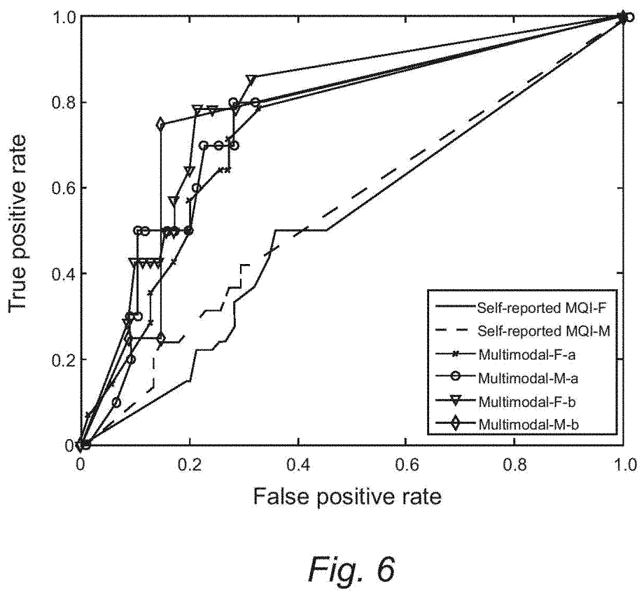

FIG. 6 shows receiver operating characteristic (ROC) curves for several feature combinations. The results showed that the area under the curve (AUC) for our multimodal indices reached up to 0.79 for females and 0.76 for males.

### DISCUSSION

The results we report here provide a proof of concept that the data collected via mobile computing methods are valid indicators of interpersonal functioning in daily life. Consistent with laboratory-based research, we found statistically significant above-chance associations between conflict behavior and several theoretically driven, individually tested data features. We also obtained significant associations between conflict and both uni-modal and multimodal feature groups with and without self-reported MQI included. In fact, our best-performing combinations of data features in several cases reached or exceeded the UA levels obtained via self-reported MQI alone.

To our knowledge, the prototype model developed for this study is the first to use machine-learning classification to identify episodes of conflict behavior in daily life using multimodal, passive computing technologies. Our study extends the literature by presenting an initial case study indicating that it is possible to detect complex psychological states using data collected in an uncontrolled environment.

Implications

Couples communicate using complex, cross-person interactional sequences where emotional, physiological, and behavioral states are shared via vocal cues and body language. Multimodal feature detection can provide a comprehensive assessment of these inter-actional sequences by monitoring the way couples react physiologically, what they say to each other, and how they say it. Couples in distressed relationships can become locked into maladaptive patterns that escalate quickly and are hard to exit once triggered. Detecting and monitoring these sequences as they occur in real time could make it possible to interrupt, alter, or even pre-vent conflict behaviors.

Thus, although preliminary, our data are an important first step toward using mobile computing methods to improve relationship functioning. The proposed algorithms could be used to identify events or experiences that precede conflict and send prompts that would decrease the likelihood that such events will spill over to affect relationship functioning. Such interventions would move beyond the realm of human-activity recognition to also include the principles of personal informatics, which help people to engage in self-reflection and self-monitoring to increase self-knowledge and improve functioning. For example, a husband who is criticized by his boss at work might experience a spike in stress levels, which could be reflected in his tone of voice, the content of his speech, and his physiological arousal. Based on this individual's pattern of arousal, our system would predict that he is at increased risk for having an argument with his spouse upon returning home that evening. A text message could be sent to prompt him to engage in a meditation exercise, guided by a computer program, that decreases his stress level. When this individual returns home, he might find that his children are arguing and that his wife is in an irritable mood. Although such situations often spark conflict between spouses, the husband might feel emotionally restored following the meditation exercise and thus be able to provide support to his wife and avoid feeling irritable himself, thereby preventing conflict.

A second option is to design prompts that are sent after a conflict episode to help individuals calm down, recover, or initiate positive contact with their partners. For example, a couple living together for the first time might get in an argument about household chores. After the argument is over, a text message could prompt each partner to independently engage in a progressive muscle relaxation exercise to calm down. Once they are in a relaxed state, the program could send a series of prompts that encourage self-reflection and increase insight about the argument—for example, what can I do to communicate more positively with my partner? What do I wish I had done differently?

In addition to detecting conflict episodes, amplifying positive moods or the frequency of positive interactions could be valuable. Potential behavioral prompts could include exercises that build upon the positive aspects of a relationship, such as complimenting or doing something nice for one's partner. Employing these methods in people's daily lives could increase the efficacy of standard therapy techniques and improve both individual and relationship functioning. Because the quality of our relationships with others plays a central role in our emotional functioning, mobile technologies thus provide an exciting approach to promoting well-being.

Limitations

Although the results from our classification experiments suggest that these methods hold promise, our findings should be interpreted in light of several limitations. Our system's classification accuracy, while moderately good given the task's inherent complexity, will need to be improved before our method can be employed widely. In our best-performing models, we missed 18 percent of conflict episodes and falsely identified 9 per-cent of cases as conflict. Classification systems that miss large numbers of conflict episodes will be limited in their ability to influence people's behaviors. At the same time, falsely identifying conflict would force people to respond to unnecessary behavior prompts, which could annoy them or cause them to discontinue use.

In our current model, classification accuracy is inhibited by several factors.

First, we relied on self-reports of conflict. Future projects could use audio recordings as an alternative, perhaps more accurate, way to identify periods of conflict.

Second, conflict and how it is experienced and expressed is highly variable across couples, with people showing different characteristic patterns in physiology or vocal tone. For example, some couples yell loudly during conflict, whereas others with-draw and become silent. One method for addressing this issue could be to train the models on individual couples during an initial trial period. By tailoring our modeling schemes, we might be able to capture response patterns specific to each person and thereby improve our classification scores.

Third, we collapsed our data into hour-long time intervals, which likely caused us to lose important information about when conflict actually started and stopped. Many conflict episodes do not last for an entire hour, and physiological responding within an hour-long period could reflect various activities besides conflict. Using a smaller time interval would likely increase accuracy.

Fourth, outside of the synchrony scores, we did not take into account the joint effects of male and female responses. Considering these together (such as male and female vocal pitch increasing at the same time) could improve our results.

Additional Studies

New algorithms have been developed to expand the types of interpersonal processes that could be detected for different constructs. In particular, an algorithm that could detect when couples were interacting with each other with 99% accuracy (balanced accuracy=0.99, kappa=0.97, sensitivity=0.98, specificity=0.99) was developed. An algorithm was then developed to detect positive mood (real, self-reported mood correlated with predicted mood at 0.75, with a mean absolute error of 14.54 on a 100-point scale). Similarly, an algorithm to detect feelings of closeness between romantic partners (real self-reported feelings of closeness between romantic partners correlated with predicted closeness at 0.85, with a mean absolute error of 11.40 on a 100-point scale). The original algorithm was also improved upon the original algorithm that detected couple conflict (from the first article). The updated algorithm was able to detect when couples were having conflict with 96% accuracy (balanced accuracy=0.96, kappa=0.90, sensitivity=0.92, specificity=0.99). Table 1 provides details of this algorithm:

TABLE 1
Conflict characterization model
Discrete

| | |
|---|---|
| Interacting | Multilayer perceptron neural network |
| Conflict | Classification trees with boosting |

Continuous

| | |
|---|---|
| Positive mood | Regression modeling using rules |
| Closeness | Random forest |

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

Amato, P. R. “The Consequences of Divorce for Adults and Children,” J. Marriage and Family, vol. 62, no. 4, 2000, pp. 1269-1287
Baucom B. R. et al., “Correlates and Characteristics of Adolescents' Encoded Emotional Arousal during Family Conflict,” Emotion, vol. 12, no. 6, 2012, pp. 1281-1291.
Burman, B., & Margolin, G. (1992). Analysis of the association between marital relationships and health problems: An interactional perspective. Psychological Bulletin, 112, 39-63. doi: 10.1037/0033-2909.112.1.39
Chaspari, T. et al., “Quantifying EDA Synchrony through Joint Sparse Representation: A Case-Study of Couples' Interactions,” Proc. Int'l Conf. Acoustics, Speech, and Signal Processing (ICASSP 15), 2015, pp. 817-821.
Coan, J. A., Schaefer, H. S., & Davidson, R. J. (2006). Lending a hand: Social regulation of the neural response to threat. Psychological Science, 17, 1032-1039. doi: 10.1111/j.1467-9280.2006.01832.x
Grewen, K. M., Andersen, B. J., Girdler, S. S., & Light, K. C. (2003). Warm partner contact is related to lower cardiovascular reactivity. Behavioral Medicine, 29, 123-130. doi: 10.1080/08964280309596065
Hung, H., & Englebienne, G. (2013). Systematic evaluation of social behavior modeling with a single accelerometer. Processing of the ACM International Joint Conference on Pervasive and Ubiquitous Computing, 127-139. doi: 10.1145/2494091.2494130
Holt-Lunstad, J., Smith, T. B., & Layton, J. B. (2010). Social relationships and mortality risk: A meta-analytic review. PLoS Medicine, 7, e1000316. doi: 10.1371/journal.pmed.1000316
House, J. S., Landis, K. R., & Umberson, D. (1988). Social relationships and health. Science, 241, 540-545.
Kim, S., Valente, F., & Vinciarelli, A. (2012). Automatic detection of conflict in spoken conversations: Ratings and analysis of broadcast political debates. Proceedings of IEEE International Conference on Acoustic, Speech, and Signal Processing, 5089-5092. doi: 10.1109/ICASSP.2012.6289065
Lawler, J. (2010). The real cost of workplace conflict. Entrepreneur. Accessed from https://www.entrepreneur.com/article/207196.
Leach, L. S., Butterworth, P., Olesen, S. C., & Mackinnon, A. (2013). Relationship quality and levels of depression and anxiety in a large population-based survey. Social Psychiatry and Psychiatric Epidemiology, 48, 417-425. doi: 10.1007/s00127-012-0559-9
Lee, Y., Min, C., Hwang, C., Lee, J., Hwang, I., Ju, Y., . . . Song J. (2013). SocioPhone: Everyday face-to-face interaction monitoring platform using multi-phone sensor fusion. Proceedings of the International Conference on Mobile Systems, Applications, and Services, 375-388. doi: 10.1145/2462456.2465426.
Levenson R. W. and Gottman, J. M., “Physiological and Affective Predictors of Change in Relationship Satisfaction,” J. Personality and Social Psychology, vol. 49, no. 1, 1985, pp. 85-94.
Lunbald, A., & Hansson, K. G. (2005). The effectiveness of couple therapy: Pre- and post-assessment of dyadic adjustment and family climate. Journal of Couple & Relationship Therapy, 4, 39-55. doi: 10.1300/J398v04n04_03
Pennebaker, J. W. et al., “The Development and Psychometric Properties of LIWC2007,” 2007; www.liwc.net/LIWC2007LanguageManual.pdf.
Robles, T. F., & Kiecolt-Glaser, J. K. (2003). The physiology of marriage: Pathways to health. Physiology and Behavior, 79, 409-416. doi: 10.1016/S0031-9384(03)00160-4
Sacker, A. (2013). Mental health and social relationships. Evidence Briefing of the Economic and Social Research Council. Accessed from http://www.esrc.ac.uk/files/news-events-and-publications/evidence-briefings/mental-health-and-social-relationships/
Simmons, R. A., Gordon, P. C., and Chambless, D. L., “Pronouns in Marital Interaction: What Do ‘You’ and ‘I’ Say about Marital Health?,” Psychological Science, vol. 16, no. 12, 2005, pp. 932-936.
Sonnentag, S., Unger, D., & Nagel, I. (2013). Workplace conflict and employee well-being: The moderating role of detachment from work during off-job time. International Journal of Conflict Management, 24, 166-183. Doi: 10.1108/10444061311316780
Springer, K. W., Sheridan, J., Kuo, D., & Carnes, M. (2007). Long-term physical and mental health consequences of childhood physical abuse: Results from a large population-based sample of men and women. Child Abuse & Neglect, 31, 517-530. doi: 10.1016/j.chiabu.2007.01.003
Timmons, A. C., Chaspari, T., Han, S. C., Perrone, L., Narayanan, S., & Margolin, G. (2017). Using multimodal wearable technology to detect conflict among couples. IEEE Computer, 50, 50-59. doi:10.1109/MC.2017.83
Timmons, A. C. Margolin, G. and Saxbe, D. E., “Physiological Linkage in Couples and Its Implications for Individual and Interpersonal Functioning,” J. Family Psychology, vol. 29, no. 5, 2015, pp. 720-731.

What is claimed is:

1. A method for monitoring and understanding interpersonal relationships comprising:
monitoring interpersonal relations of a couple or group of interpersonally connected users with a plurality of smart devices by collecting data streams from the smart devices or from wearable sensor in communication with the smart devices;
classifying and/or quantifying the interpersonal relations into classification or quantifications; and
providing feedback and/or goals to one or more users to increase awareness about relationship functioning.

2. The method of claim 1 wherein representations of interpersonal relationships are formed for increasing knowledge about relationship functioning and detecting interpersonally-relevant mood states and events.

3. The method of claim 2 wherein the representations of interpersonal relationships are signal-derived and machine-learned representations.

4. The method of claim 1 wherein signal-derived features are extracted from the data streams, and the signal derived features providing inputs to a trained neural network are interpersonal classifications that allow selection of a predetermined feedback to be sent.

5. The method of claim 1 wherein the data streams include one or more components selected from the group consisting of physiological signals, audio measures, speech content, video, GPS, light exposure, content consumed and exchanged through mobile, internet, network communications, sleep characteristics, interaction measures between individuals and across channels, and self-reported data about relationship quality, negative and positive interactions, and mood.

6. The method of claim 5 wherein pronoun use, negative emotion words, swearing, certainty words in speech content are evaluated.

7. The method of claim 4 wherein content of text messages and emails, time spent on the internet, number or length of texts and phone calls in network communications are measured.

8. The method of claim 1 wherein data or the data streams are stored separately in a peripheral device or integrated into a single platform.

9. The method of claim 8 wherein the peripheral device is a wearable sensor, cell phone, or audio storage device.

10. The method of claim 8 wherein the single platform is a mobile device or IoT platform.

11. The method of claim 1 further comprising computing signal-derived features of the data streams.

12. The method of claim 11 wherein the signal-derived features are computed by knowledge-based feature design and/or data-driven clustering.

13. The method of claim 11 wherein the signal-derived features are used as a foundation for machine learning, data mining, and statistical algorithms that are used to determine what factors, or combination of factors, predict a variety of relationship dimensions, such as conflict, relationship quality, or positive interactions

14. The method of claim 1 wherein individualized models increase classification accuracy, since patterns of interaction may be specific to individuals, couples, or groups of individuals.

15. The method of claim 1 where active and semi-supervised learning are applied to increase predictive power as people continue to use a system implementing the method.

16. The method of claim 1 wherein the relationship functioning includes indices selected from the group consisting of a ratio of positive to negative interactions, number of conflict episodes, an amount of time two users spent together, an amount of quality time two users spent together, amount of physical contact, exercise, time spent outside, sleep quality and length, and coregulation or linkage across these measures.

17. The method of claim 16 wherein further comprising suggesting goals for these indices and allows users to customize their goals.

18. The method of claim 1 wherein feedback is provided as ongoing tallies and/or graphs viewable on the smart devices.

19. The method of claim 1 further comprising creating daily, weekly, monthly, and yearly reports of relationship functioning.

20. The method of claim 1 further comprising allowing users to view, track, and monitor each of these data streams and their progress on their goals via customizable dashboards.

21. The method of claim 1 further comprising analyzing each data stream to provide a user with covariation of user's mood, relationship functioning, and various relationship-relevant events.

22. The method of claim 1 wherein user can create personalized networks and specify relationship types for each person in their network.

23. The method of claim 1 wherein users set person-specific privacy settings and customize personal data that can be accessed by others in their networks.

24. A system comprising a plurality of mobile smart devices operated by a plurality of users wherein at least one smart device or a combination of smart devices execute steps of:
monitoring interpersonal relations of a couple or group of interpersonally connected users with a plurality of smart devices by collecting data streams from the smart devices;
classifying and/or quantifying the interpersonal relations; and
providing feedback and/or goals to one or more users to increase awareness about relationship functioning, the system including:

25. The system of claim 24 further comprising a plurality of sensors worn by the couple or group of interpersonally connected users.

26. The system of claim 24 wherein the mobile smart devices include a microprocessor and non-volatile memory on which instructions for implementing the method are stored.
